(19) United States
(12) Patent Application Publication
(10) Pub. No.: US 2026/0345488 A1
(43) Pub. Date: Nov. 5, 2026

(54) PHARMACEUTICAL FORMULATIONS

(71) Applicant: Gelteq Limited, Caulfield (AU)

(72) Inventors: Nathan Jacob Givoni, Caulfield (AU); Julie Huynh, Caulfield (AU); Paul Michael Wynne, Caulfield (AU); Kristina Tolotchkov, Caulfield (AU)

(21) Appl. No.: 19/486,918

(22) PCT Filed: May 21, 2024
(86) PCT No.: PCT/IB2024/054921

§ 371 Date: Nov. 21, 2025

(30) Foreign Application Priority Data
May 22, 2023 (GB) 2307626.8

(51) Int. Cl. A61K 9/00; A61K 9/06; A61K 31/192; A61K 31/196; A61K 31/549; A61K 47/02; A61K 47/12; A61K 47/36

(52) U.S. Cl. CPC A61K 9/0095; A61K 9/06; A61K 31/192; A61K 31/196; A61K 31/549; A61K 47/02; A61K 47/12; A61K 47/36

(57) ABSTRACT

A drinkable gel formulation for a Biopharmaceutics Classification System (BCS) class II Active Pharmaceutical Ingredient (API) is disclosed. The drinkable gel formulation includes a Biopharmaceutics Classification System (BCS) class II Active Pharmaceutical Ingredient (API), or a salt or solution thereof; a combination of gelling agents including: a primary gelling agent which is agar, a secondary gelling agent which is carrageenan, and a tertiary gelling agent which is an alginate; at least one cation donator; a pH modifier; a preservative; and water.

This invention relates to pharmaceutical formulations and more particularly to one for an Active Pharmaceutical Ingredient (API) which is recognised under the Biopharmaceutics Classification System (BCS) as a Class II drug.

### BACKGROUND

API's can be difficult to formulate as their properties differ from API to API. Two of the parameters that challenge a formulation chemist are an API's solubility and its permeability. Accordingly, API's are broadly defined as those falling into one of four classification types based on these two parameters. These four types are illustrated in FIG. 1.

Class I API's have high permeability and high solubility.
a. An Examples is Metoprolol.
b. Class I compounds are well absorbed, and their absorption rate is usually higher than excretion.

Class II API's have high permeability and low solubility.
a. Examples include Ibuprofen and Naproxen.
b. The bioavailability of those products is limited by their solvation rate. A correlation between the in vivo bioavailability and the in vitro solvation can be found.

Class III API's have low permeability and high solubility.
a. An example is Cimetidine.
b. The absorption is limited by the permeation rate, but the drug is solvated very fast. If the formulation does not change the permeability or gastro-intestinal duration time, then class I criteria can be applied.

Class IV API's have low permeability and low solubility.
a. An example is Bifonazole.
b. These compounds have a poor bioavailability. Usually, they are not well absorbed over the intestinal mucosa and a high variability is expected.

The API's are classified in BCS on the basis of solubility, permeability, and also dissolution.

Solubility class boundaries are based on the highest dose strength of an immediate release product. A drug is considered highly soluble when the highest dose strength is soluble in 250 ml or less of aqueous media over the pH range of 1 to 7.5. The volume estimate of 250 ml is derived from typical bioequivalence study protocols that prescribe administration of a drug product to fasting human volunteers with a glass of water.

Permeability class boundaries are based indirectly on the extent of absorption of a drug substance in humans and directly on the measurement of rates of mass transfer across human intestinal membrane. Alternatively non-human systems capable of predicting drug absorption in humans can be used (such as in-vitro culture methods). A drug substance is considered highly permeable when the extent of absorption in humans is determined to be 90% or more of the administered dose based on a mass-balance determination or in comparison to an intravenous dose.

Dissolution class boundaries include an immediate release product, namely one which is rapidly dissolving, i.e. when no less than 85% of the labelled amount of the drug substance dissolves within 15 minutes using USP Dissolution Apparatus 1 at 100 RPM or Apparatus 2 at 50 RPM in a volume of 900 ml or less in the following media: 0.1 M HCl or simulated gastric fluid or pH 4.5 buffer and pH 6.8 buffer or simulated intestinal fluid.

Most oral formulations take the form of filled capsules or tablets.

Other formulation types are known, and Applicant has developed a number of drinkable gel-based formulations in areas such as glucose tolerance testing and for delivering nutraceuticals-see respectively: WO2017/075672 and WO2019215641.

It is an object of the present invention to provide alternative pharmaceutical formulations for BCS Class II drugs as exemplified by, for example, Ibuprofen and Naproxen.

Ibuprofen belongs to the drug class called nonsteroidal anti-inflammatory drugs (NSAIDs).

It comes in various oral dosage forms including oral tablets, oral capsules, oral suspension and chewable tablets.

Branded Ibuprofens include:
US: Equate, Advil, and Motrin IB;
AU: Nurofen, Wagner Health, and Rafen; and
UK: Boots, Nurofen, Calprofen, Ibuleve, Cuprofen, and Flarin

Naproxen is also a NSAID.

It comes in various oral dosage forms including tablets, enteric coated tablets, suspensions and liquid filled capsules.

Branded Naproxens include:
Aflaxen, Aleve, Aleve Arthritis, Anaprox, Anaprox DS, EC Naprosyn, Naprelan, Naprelan 500, Naprelan Dose Card and Naprosyn.

From a regulatory perspective it is possible to obtain registration for a new dosage form using an abridged process such as that provided by the FDA using 505 (B)(2) regulatory pathways. See for example: https://www.fda.gov/media/156350/download incorporated by reference.

Dosage forms identified in prior art searches include:

CN 110833561 which discloses a chewable tablet comprising the API omeprazole and a gel component comprising carrageenan, sodium alginate, and agar in amounts of 3-5:2-3:1.

CN 111437261 which discloses a chewable tablet comprising the API idicalcidol and a gel component comprising carrageenan, sodium alginate, and agar in amounts of 3-5:2-3:1.

US 2008/0160087 which discloses a gel formulation comprising fluvastatin and a combination of gelling agents to improve taste and address syneresis.

US 2015/0174247 which discloses an oral pharmaceutical preparation comprising aripiprazole and one or more gelling agents.

U.S. Pat. No. 6,106,867 which discloses a propolis food product comprising gelling agents; and

JP 2000302670 which discloses a carrageenan-based ibuprofen formulation further comprising locust bean gum and xanthan gum. It is an object of the present invention to develop alternative dosage forms for BSC class II drugs as exemplified with reference to Ibuprofen and Naproxen, and in particular “drinkable” gel-based formulations which can be targeted at patient groups that find swallowing tablets difficult, such as the elderly and children.

### BRIEF SUMMARY OF THE DISCLOSURE

In accordance with the present inventions there is provided a drinkable gel formulation for a Biopharmaceutics Classification System (BCS) class II Active Pharmaceutical Ingredient (API) comprising:
i) a Biopharmaceutics Classification System (BCS) class II Active Pharmaceutical Ingredient (API), or a salt or solution thereof;
ii) a combination of gelling agents comprising of:
   a. a primary gelling agent which is agar,
   b. a secondary gelling agent which is carrageenan, and
   c. a tertiary gelling agent which is an alginate;
iii) at least one cation donator;
iv) a pH modifier;
v) a preservative; and
vi) water.

Preferably, the BCS class II formulation further comprises:
vii) a flavouring.

The terms “primary”, “secondary” and “tertiary” are used to denote the relative proportion (by weight percent) of the respective gelling agents such that the primary gelling agent is present in the greatest amount and the tertiary gelling agent is present in the least amount relative to one another.

The selection of agar as the primary gelling agent is of significance to the formulation being a “drinkable” gel since the agar releases water when subjected to a shear force when it is sucked, thereby facilitating it's uptake without “chewing” distinguishing the formulation from “edible” gels.

Non-limiting examples of formulations comprising a BCS class II API include formulations in which the API is ibuprofen or naproxen, or a salt or solution thereof.

Preferred ibuprofen or naproxen formulations are sodium salts or solutions thereof.

Other exemplary BCS class II API's include:
Hydrochlorothiazide and Diclofenac.
Hydrochlorothiazide is a diuretic medication often used to treat high blood pressure.
Diclofenac is another non-steroidal anti-inflammatory.

The alternative gel formulations of the invention comprise agar as a primary gelling agent, as on sucking it shears to release water making the delivery system particularly attractive as a delivery means for children and the elderly who find swallowing tablets difficult. However, in order to accommodate different class II API's, provide dissolution, stability and texture Applicant determined that both specific secondary and tertiary gelling agents were additionally required.

As noted above the primary gelling agent is Agar.

Agar is outstanding among hydrocolloids. Agar-agar gels can be formed in very dilute solutions, containing as little as 0.5% to 1.0% of agar-agar. These gels are rigid, brittle, have well defined shapes, as well as sharp melting and gelling points. Moreover, they clearly demonstrate the interesting phenomenon of syneresis (spontaneous extrusion of water through the surface of the gel), and hysteresis (temperature interval between melting and gelling temperatures). Gelling occurs at temperatures far below the gel melting temperature. A 1.5% solution of agar-agar forms a gel on cooling to about 32° to 45 °C that does not melt below 85 °C. This hysteresis interval is a novel property of agar-agar that finds many uses in food applications. The gel strength of the agar-agar is influenced by concentration, time, pH, and sugar content. The pH noticeably affects the strength of the agar gel; as the pH decreases, the gel strength weakens. Sugar content also has a considerable effect over agar gel. Increasing levels of sugar make gels with harder but a less cohesive texture.

The secondary agent is a carrageenan, more particularly kappa carrageenan.

Carrageenan's are large, highly flexible molecules that form curling helical structures. This gives them the ability to form a variety of different gels at room temperature. They are widely used in the food and other industries as thickening and stabilizing agents.

All carrageenan's are high-molecular-weight polysaccharides and are mainly made up of alternating 3-linked β-D-galac-topyranose (G-units) and 4-linked α-D-galactopyranose (D-units) or 4-linked 3,6-anhydro--D-galactopyranose (DA-units), forming the disaccharide repeating unit of carrageenan's.

There are three main commercial classes of carrageenan:
Kappa which forms strong, rigid gels in the presence of sodium and potassium ions.
lota which forms soft gels in the presence of calcium ions, and
Lambda which does not gel.

The tertiary gelling agent is an alginate. The alginates include:
Sodium alginate (NaC₅H/O₆),
Potassium alginate (KC₆H₇O₆), and
Calcium alginate (CaC₁₂H₁₄O₁₂).

The three gelling agents are optimally used in the relative proportions (by weight) of about: Agar: Carrageenan: Alginate of 4.5-2.6:2.5-1.5:1 more preferably, 4-3:2.5-1.5:1.

The preferred gelling agents are:
a. agar-agar,
b. kappa carrageenan, and
c. sodium alginate.

To facilitate gelling one or more cation donators are judicially selected.

Preferably the
iii) at least one cation donator is one of a divalent or monovalent cation, more preferably a calcium, magnesium, or potassium salt. Preferred salts include magnesium and potassium chloride.

In a preferred embodiment the
iii) cation donator consists or comprises potassium chloride.
Preferably the
iv) the at least one pH modifier is an acidifying agent, alkalizing agent or buffering agent. Exemplary modifiers include: acetic acid, citric acid, hydrochloric acid, phosphoric acid and sodium hydroxide.

In a preferred embodiment the formulation comprising a BCS class II API has as the acidifying agent citric acid.

The citric acid may be used in an amount of up to 2.88% (by weight).
Preferably the
v) at least one preservative is selected from lactic acid, potassium sorbate and sodium benzoate.

In a preferred embodiment the formulation comprising a BCS class II API has as
v) the at least one preservative, potassium sorbate.

The potassium sorbate may be used in an amount of from 0.07 to 0.12%

Generally, for a formulation comprising a BCS class II API the gelling agents comprise a primary, secondary, and tertiary gelling agent comprising respectively, by weight percent of the total formulation as claimed:
a. agar-0.47%-0.95%,
b. carrageenan-0.31%-0.47%, and
c. an alginate-0.16%-0.24%.

With respect to the weight percent figures, these are based on the essential components specified in the claims and exclude optional excipients such as a favouring which may, for example, be added in amounts of up to 2% by weight.

The actual amounts vary with the API selected.

In a first embodiment, where the API is Ibuprofen, the gelling agents are present in an amount by weight of:
a. agar-0.47%-0.71%,
b. carrageenan-0.31%-0.47%, and
c. an alginate-0.16%-0.24%.

More particularly the formulation is as illustrated in Table 1 below:

TABLE 1

| Excipient | Weight percent (Range) | Mean |
|---|---|---|
| Ibuprofen (sodium | 0.69-1.03% | 0.86% |
| salt) | | |
| Water | ≥78.18% | 97.7% |
| Potassium sorbate | 0.078%-0.117% | 0.1% |
| Magnesium | 0.034%-0.052% | 0.04% |
| chloride | | |
| Potassium chloride | 0.016%-0.024% | 0.02% |
| Agar | 0.47%-0.70% | 0.59% |
| Kappa | 0.31%-0.47% | 0.39% |
| Carrageenan | | |
| Sodium alginate | 0.16%-0.24% | 0.20% |
| Citric acid | 0.066%-0.099% | 0.08% |
| TOTAL | | 100% |

Actual excipient amounts are illustrated in Table 2a and 2b below:

TABLE 2a
Ibuprofen gel (200 mg/25 ml) - pH 6.8

| INGREDIENT | MASS | UNIT | % |
|---|---|---|---|
| Ibuprofen | 0.200 | g | 0.78% |
| Water | 25 | g | 97.80% |
| Potassium | 0.025 | g | 0.10% |
| sorbate | | | |
| Magnesium | 0.011 | g | 0.04% |
| chloride | | | |
| Potassium | 0.005 | g | 0.02% |
| chloride | | | |
| Agar | 0.150 | g | 0.59% |
| Kappa | 0.100 | g | 0.39% |
| Carrageenan | | | |
| Sodium alginate | 0.050 | g | 0.20% |
| Citric acid | 0.021 | g | 0.08% |
| TOTAL | 25.562 | g | 100.00% |

TABLE 2b
Ibuprofen gel (220 mg/25 ml) - pH 6.8

| INGREDIENT | MASS | UNIT | % |
|---|---|---|---|
| Ibuprofen | 0.220 | g | 0.86% |
| Water | 25 | g | 97.72% |
| Potassium sorbate | 0.025 | g | 0.10% |
| Magnesium chloride | 0.011 | g | 0.04% |
| Potassium chloride | 0.005 | g | 0.02% |
| Agar | 0.150 | g | 0.59% |
| Kappa Carrageenan | 0.100 | g | 0.39% |
| Sodium alginate | 0.050 | g | 0.20% |
| Citric acid | 0.021 | g | 0.08% |
| TOTAL | 25.582 | g | 100.00% |

In a second embodiment, where the API is Naproxen, the gelling agents are present in an amount by weight of:
a. agar-0.63%-0.95%,
b. carrageenan-0.31%-0.47%, and
c. an alginate-0.16%-0.24%.

More particularly the formulation is as illustrated in Table 3 below:

TABLE 3

| Excipient | Weight percent (Range) | Mean |
|---|---|---|
| Naproxen | 0.69%-1.03% | 0.86% |
| Water | ≥78.12% | 97.7% |
| Potassium | 0.08%-0.12% | 0.10% |
| sorbate | | |
| Potassium | 0.16%-0.23% | 0.20% |
| chloride | | |
| Agar | 0.63%-0.94% | 0.78% |
| Kappa | 0.31%-0.47% | 0.39% |
| Carrageenan | | |
| Sodium alginate | 0.16%-0.23% | 0.20% |
| Citric acid | 0.0009%-0.0014% | 0.0012% |
| TOTAL | | 100% |

Actual excipient amounts are illustrated in Table 4 below:

TABLE 4
Naproxen gel (220 mg/25 ml) - pH 7.5

| INGREDIENT | MASS | UNIT | % |
|---|---|---|---|
| Naproxen | 0.22 | g | 0.86% |
| Water | 25.00 | g | 97.66% |
| Potassium | 0.025 | g | 0.10% |
| sorbate | | | |
| Potassium | 0.005 | g | 0.02% |
| chloride | | | |
| Agar | 0.200 | g | 0.78% |
| Kappa | 0.100 | g | 0.39% |
| Carrageenan | | | |
| Sodium alginate | 0.050 | g | 0.20% |
| Citric acid | 0.0003 | g | 0.0012% |
| TOTAL | 25.60 | g | 100.00% |

In a third embodiment, where the API is Diclofenac, the gelling agents are present in an amount by weight of:
a. agar-0.47%-0.71%,
b. carrageenan-0.32%-0.47%, and
c. an alginate-0.16%-0.24%.

More particularly the formulation is as illustrated in Table 5 below:

TABLE 5

| Excipient | Weight percent (Range) | Mean |
|---|---|---|
| Diclofenac | 0.038%-0.057% | 0.05% |
| Water | ≥78.88% | 98.6% |
| Potassium sorbate | 0.076-0.114% | 0.09% |
| Potassium chloride | 0.015-0.023% | 0.02% |
| Agar | 0.53-0.79% | 0.66% |
| Kappa | 0.30-0.46% | 0.38% |
| Carrageenan | | |
| Sodium alginate | 0.15-0.23% | 0.19% |
| Citric acid | 0.0012-0.0018% | 0.0015% |
| TOTAL | | 100% |

Actual excipient amounts are illustrated in Table 6 below:

TABLE 6
Diclofenac gel (12.5 mg/25 ml) - pH 7.5

| INGREDIENT | MASS | UNIT | % |
|---|---|---|---|
| Diclofenac | 0.0125 | g | 0.05% |
| Water | 26.00 | g | 98.60% |
| Potassium | 0.025 | g | 0.09% |
| sorbate | | | |
| Potassium | 0.005 | g | 0.02% |
| chloride | | | |
| Agar | 0.175 | g | 0.66% |
| Kappa | 0.100 | g | 0.38% |
| Carrageenan | | | |
| Sodium | 0.050 | g | 0.19% |
| alginate | | | |
| Citric acid | 0.0004 | g | 0.0015% |
| TOTAL | 26.3679 | g | 100.00% |

In a fourth embodiment, where the API is Hydrochlorothiazide, the gelling agents are present in an amount by weight of:
a. agar-0.47%-0.71%,
b. carrageenan-0.32%-0.47%, and
c. an alginate-0.16%-0.24%.

More particularly the formulation is as illustrated in Table 7 below:

TABLE 7

| Excipient | Weight percent (Range) | Mean |
|---|---|---|
| Hydrochlorothiazide | 0.038-0.057% | 0.05% |
| Water | ≥78.9% | 98.65% |
| Potassium sorbate | 0.076-0.11% | 0.10% |
| Potassium chloride | 0.015-0.023% | 0.02% |
| Magnesium | 0.034-0.050% | 0.04% |
| chloride | | |
| Agar | 0.46-0.69% | 0.57% |
| Kappa | 0.31-0.46% | 0.38% |
| Carrageenan | | |
| Sodium alginate | 0.15-0.23% | 0.19% |
| Citric acid | 0.0012-0.0018% | 0.0015% |
| TOTAL | | 100% |

Actual excipient amounts are illustrated in Table 8 below:

TABLE 8
Hydrochlorothiazide (12.5 mg/25 ml) - pH 7.5

| INGREDIENT | MASS | UNIT | % |
|---|---|---|---|
| Hydrochlorothiazide | 0.0125 | g | 0.05% |
| Water | 25.87 | g | 98.65% |
| Potassium sorbate | 0.025 | g | 0.10% |
| Potassium chloride | 0.005 | g | 0.02% |
| Magnesium chloride | 0.011 | g | 0.04% |
| Agar | 0.150 | g | 0.57% |
| Kappa Carrageenan | 0.100 | g | 0.38% |
| Sodium alginate | 0.050 | g | 0.19% |
| Citric acid | 0.0004 | g | 0.0015% |
| TOTAL | 26.2239 | g | 100.00% |

The invention is further described, by way of example only, with reference to the drawings, Examples and detailed description given below.

### BRIEF DESCRIPTION OF THE DRAWINGS

Embodiments of the invention are further described hereinafter with reference to the accompanying drawings, in which:

FIG. 1 is a diagram illustrating the Biopharmaceutics Classification System (BCS);

FIG. 2 is a graph illustrating the dissolution of five individual gels; and

FIGS. 3a and 3b are graphs illustrating the dissolution of the preferred triple combination with ibuprofen at 200 mg and 220 mg respectively vs Advil and Motrin (alternative dosage forms).

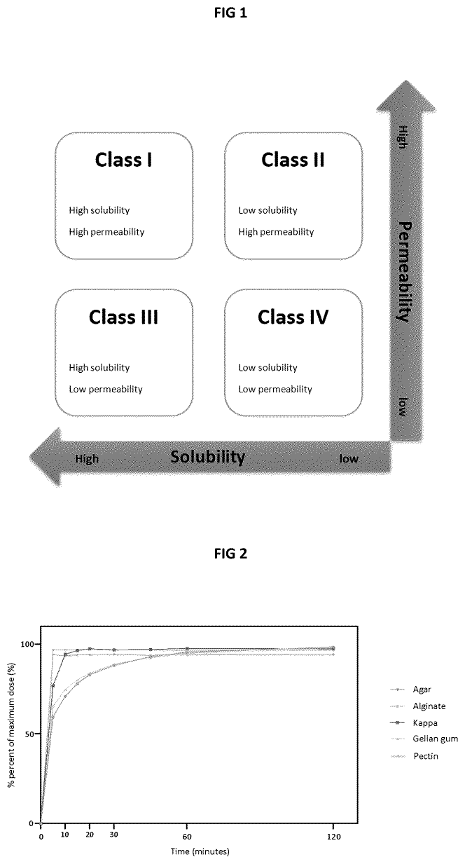

### DETAILED DESCRIPTION

In developing alternative formulations for BCS class II API formulations Applicant looked at a variety of standard gelling agents with a first Exemplary API (Ibuprofen).

### Experimental Series 1

From a stock solution of ibuprofen (Table 9 below), a number of mono-gel formulations were produced, and dissolution was tested.

TABLE 9
(1) Ibuprofen sodium stock solution (Ibu-Na solution)

| Ingredient | Mass | Unit | % |
|---|---|---|---|
| Ibuprofen | 1.20 | g | 0.79 |
| Sodium hydroxide | 0.23 | g | 0.15 |
| Water | 150.00 | g | 99.06 |

Using this stock solution gels the following mono gels were produced and the formulations are provided in Tables 10 to 14:

### 1) Agar

TABLE 10

| | |
|---|---|
| Excipients | Agar only |

| Unit | g | % |
|---|---|---|
| Ibu-Na soln | 25 | 99.16 |
| Magnesium chloride | 0.011 | 0.04 |
| Potassium sorbate | 0.01 | 0.04 |
| Agar | 0.19 | 0.75 |

2) Kappa carrageenan,

TABLE 11

| | |
|---|---|
| Excipients | Kappa Carrageenan only |

| Unit | g | % |
|---|---|---|
| Ibu-Na soln | 25 | 99.1 |
| Magnesium chloride | 0.011 | 0.04 |
| Potassium sorbate | 0.01 | 0.04 |
| Potassium chloride | 0.005 | 0.02 |
| Kappa carrageenan | 0.2 | 0.79 |

3) Low acyl gellan gum

TABLE 12

| | |
|---|---|
| Excipients | Low acyl gellan gum only |

| Unit | g | % |
|---|---|---|
| Ibu-Na soln | 25 | 99.12 |
| Magnesium chloride | 0.011 | 0.04 |
| Potassium sorbate | 0.01 | 0.04 |
| Low acyl gellan gum | 0.2 | 0.79 |

4) Sodium alginate

TABLE 13

| | |
|---|---|
| Excipients | Sodium alginate only |

| Unit | g | % |
|---|---|---|
| Ibu-Na soln | 25 | 99.52 |
| Magnesium chloride | 0.011 | 0.04 |
| Potassium sorbate | 0.01 | 0.04 |
| Sodium alginate | 0.1 | 0.40 |
| Notes | Formulation | |
| | remained as a | |
| | liquid | |

### 5 5) Pectin

TABLE 14

| | |
|---|---|
| Excipients | Pectin only |

| Unit | g | % |
|---|---|---|
| Ibu-Na soln | 25 | 99.52 |
| Magnesium chloride | 0.011 | 0.04 |
| Potassium sorbate | 0.01 | 0.04 |
| Pectin | 0.1 | 0.40 |
| Notes | Formulation | |
| | remained as a | |
| | liquid | |

### Results

The dissolution data for these formulations are provided in FIG. 2.

As can be seen from FIG. 2, the results showed the “gels” to perform differently.

The two “gels”, which did not actually gel in the given formulations, namely-sodium alginate (red circle) and pectin (blue diamond), perhaps unsurprisingly given their state, showed the most rapid dissolution, releasing their full dose inside of 5 minutes.

On the other hand, Agar (yellow diamond) and Gellan gum (pink triangle) took about 60 minutes to fully release the Ibuprofen.

Surprisingly, the Kappa carrageenan, which forms gels via ionic association with potassium ions, produced the fastest dissolution profile with 0.02% potassium chloride, releasing the full dose of ibuprofen within 5 minutes. The gel had a soft and bouncy texture and therefore fully disintegrated. Although this is great for a therapeutic it proved not to be temperature stable, and therefore a second series of experiments (Experimental series 2) looked at utilising the kappa carrageenan and modifying it with other gelling agent(s) to improve its stability (and texture).

### Experimental Series 2

To strengthen the kappa carrageenan gel base, agar was introduced. In contrast to kappa carrageenan, agar forms gels via cold-set gelation. Agar was dissolved and dispersed in boiling water. Upon cooling, inter-chain helices form between the polymers, leading to a three-dimensional network. The introduction of agar with kappa carrageenan produced semi-solid gels compared to kappa carrageenan alone. However, the gels remained soft and bouncy, rather than rigid and firm.

Concentrations of agar from 0.20-0.32% resulted in a soft gel which crumbled when crushed. Agar concentrations of 0.44-0.55% resulted in a firmer gel that broke apart when squished. When the kappa carrageenan concentration changed from 0.28-0.75%, the gel texture was soft but there was no noticeable difference. Therefore, contrary to the indications from Experimental series 1, agar became the primary gelling agent and kappa the secondary gelling agent.

An exemplary, two gel combination is provided in Table 15 below:

TABLE 15
Kappa & Agar

| Ingredient | Mass | % | Unit |
|---|---|---|---|
| Ibu-Na soln | 25 | 99.10% | g |
| Magnesium | 0.011 | 0.04% | g |
| chloride | | | |
| Potassium | 0.01 | 0.04% | g |
| sorbate | | | |
| Potassium | 0.005 | 0.02% | g |
| chloride | | | |
| Kappa | 0.09 | 0.36% | g |
| carrageenan | | | |
| Agar agar | 0.11 | 0.44% | g |
| TOTAL | 25.226 | 100.00% | g |

| Observations | Semi-clear gel, firm, holds shape. clean |
|---|---|
| | interior cut. good texture gel when |
| | squished, collapses under the spatula |
| | but not too soft |

However, the two gelling agent composition, whilst having suitable dissolution and stability characteristics, had a texture which was unsatisfactory. Therefore, a third gelling agent was introduced-Series 3.

### Experimental Series 3

Following extensive experimentation Applicant determined that sodium alginate should be added, and a desirable formulation was obtained that was temperature stable.

An exemplary formulation is illustrated in Table 16 below:

TABLE 16
Kappa, Agar & Alginate

| Ingredient | Mass | % | Unit |
|---|---|---|---|
| Ibu-Na soln | 25 | 98.71 | g |
| Magnesium | 0.011 | 0.04 | g |
| chloride | | | |
| Potassium | 0.01 | 0.04 | g |
| sorbate | | | |
| Potassium | 0.005 | 0.02 | g |
| chloride | | | |
| Kappa | 0.1 | 0.39 | g |
| carrageenan | | | |
| Agar agar | 0.15 | 0.59 | g |
| Sodium | 0.05 | 0.20 | g |
| alginate | | | |
| TOTAL | 25.326 | 100.00 | |

| | |
|---|---|
| Observations | Firm gel, homogenous, cloudy appearance due to |
| | gelling agents. Clean cut. Collapses when squished |
| | (not pasty) |

However, the ibuprofen sodium solution had a pH of 9.90. Therefore, a series of experiments were undertaken to modify the pH.

Hydrochloric acid and citric acid were tested as pH adjustors. Hydrochloric acid precipitated and was not compatible with the formulation. Citric acid changed the pH significantly. As a consequence, it was selected as the preferred pH adjusting agent. Further details of the ibuprofen gels of the invention are set out in Example 1.

### Example 1 (Ibuprofen)

The preferred Ibuprofen formulations are set out in Tables 2a and 2b reproduced below:

TABLE 2a
Ibuprofen gel (200 mg/25 ml) - pH 6.8

| INGREDIENT | MASS | UNIT | % |
|---|---|---|---|
| Ibuprofen | 0.200 | g | 0.78% |
| Water | 25 | g | 97.80% |
| Potassium | 0.025 | g | 0.10% |
| sorbate | | | |
| Magnesium | 0.011 | g | 0.04% |
| chloride | | | |
| Potassium | 0.005 | g | 0.02% |
| chloride | | | |
| Agar | 0.150 | g | 0.59% |
| Kappa | 0.100 | g | 0.39% |
| Carrageenan | | | |
| Sodium alginate | 0.050 | g | 0.20% |
| Citric acid | 0.021 | g | 0.08% |
| TOTAL | 25.562 | g | 100.00% |

TABLE 2b
Ibuprofen gel (220 mg/25 ml) - pH 6.8

| INGREDIENT | MASS | UNIT | % |
|---|---|---|---|
| Ibuprofen | 0.220 | g | 0.86% |
| Water | 25 | g | 97.72% |
| Potassium sorbate | 0.025 | g | 0.10% |
| Magnesium chloride | 0.011 | g | 0.04% |
| Potassium chloride | 0.005 | g | 0.02% |
| Agar | 0.150 | g | 0.59% |
| Kappa Carrageenan | 0.100 | g | 0.39% |
| Sodium alginate | 0.050 | g | 0.20% |
| Citric acid | 0.021 | g | 0.08% |
| TOTAL | 25.582 | g | 100.00% |

The selected ibuprofen gel formulation was modified to produce a product with “bio equivalence” to an approved ibuprofen formulation so that the FDA 505 pathway could be used to expedite approval.

### Formulating the Gel

To prepare the ibuprofen solution, dissolve sodium hydroxide pellets in water. Add ibuprofen powder slowly until completely dissolved. Add citric acid to obtain a pH~7. Using a stirring hot plate, heat the ibuprofen solution to 95 °C. Add agar and mix until completely dissolved and clear. Add potassium sorbate, magnesium chloride, potassium chloride and mix until dissolved. Add citric acid and mix. Slowly add the remaining gelling agents, kappa carrageenan and sodium alginate and mix until completely dissolved. Hot fill the gel into the pouch and cap immediately. Let it cool to room temperature. Pouches may require air to inflate the pouch prior to filling.

Preparing the gel for dissolution testing Once cool and set, squeeze the pouch 5 times prior to opening.

### Dissolution Test

Fill dissolution vessel with 900 mL medium (phosphate-buffered saline, PBS pH 7.2). Using the paddle attachment, set rotating speed at 50 rpm and temperature of the water bath at 37.5 °C. Dispense squished gel from the pouch into the dissolution vessel and start experiment. Sample buffer medium at the following time points: 5, 10, 15, 20, 30, 45, 60, 120 minutes and filter immediately after sampling. Replace removed buffer after sampling.

### High Performance Liquid Chromatography

The HPLC was performed as set out in Table 17 below:

TABLE 17

| Parameter | Value | |
|---|---|---|
| Instrument | Shimadzu Nexera X2 UHPLC system attached to Shimadzu SPD-M30A | |
| | Diode array detector and Shimadzu CTO-20AC column oven | |
| Column | Phenomenex Kinetex ® 2.6 μm C18 100 Å (100 × 2.1 mm) LC Column | |
| UV Wavelength | 220 nm | |
| Mobile Phase | 0.1% formic acid in H₂O | |
| A | | |
| Mobile Phase | 0.1% formic acid in ACN | |
| B | | |
| Flow rate | 0.5 mL/min | |
| | Time (min) | % Mobile phase B |
| Gradient | 0 | 20 |
| | 0.05 | 20 |
| | 5 | 90 |
| | 6 | 90 |
| | 7 | 20 |
| | 10 | 20 |
| Temperature | 40 °C | |
| Injection | 1 μL | |
| volume | | |
| Peak retention | 4.28 min | |
| time | | |
| Concentration | 50-300 μg/mL | |
| range | | |

### Validation

The assay was validated based on replicates at three concentrations and were considered valid when precision and accuracy were within +10%.

### Results

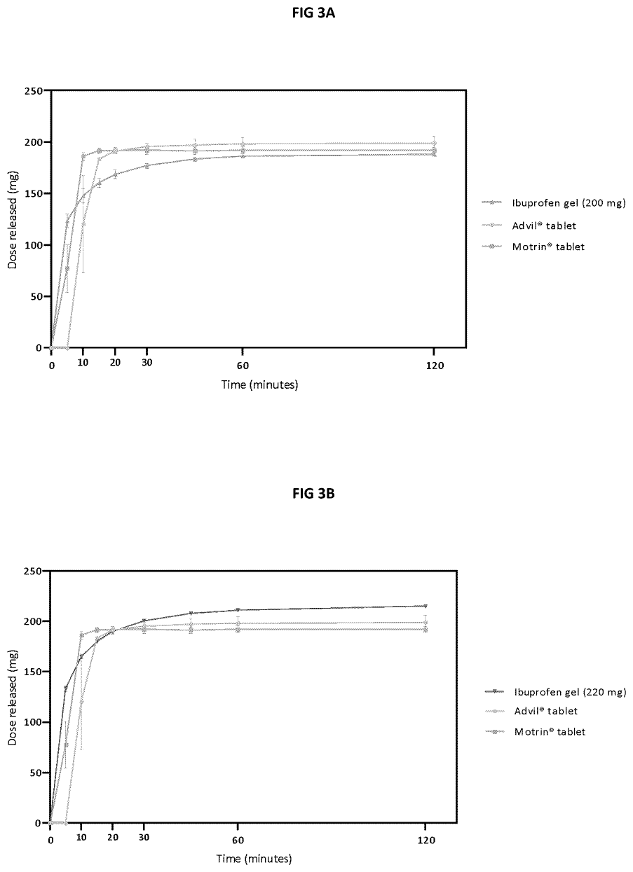

FIGS. 3a and 3b show the dissolution profile of a 200 mg and 220 mg formulation of ibuprofen respectively against formulations of Advil and Motrin respectively.

### Example 2 (Naproxen)

TABLE 4
Naproxen gel (220 mg/25 ml) - pH 7.5

| INGREDIENT | MASS | UNIT | % |
|---|---|---|---|
| Naproxen | 0.22 | g | 0.86% |
| Water | 25.00 | g | 97.66% |
| Potassium | 0.025 | g | 0.10% |
| sorbate | | | |
| Potassium | 0.005 | g | 0.02% |
| chloride | | | |
| Agar | 0.200 | g | 0.78% |
| Kappa | 0.100 | g | 0.39% |
| Carrageenan | | | |
| Sodium alginate | 0.050 | g | 0.20% |
| Citric acid | 0.0003 | g | 0.0012% |
| TOTAL | 25.60 | g | 100.00% |

### Example 3 (Diclofenac)

TABLE 6
Diclofenac gel (12.5 mg/25 ml) - pH 7.5

| INGREDIENT | MASS | UNIT | % |
|---|---|---|---|
| Diclofenac | 0.0125 | g | 0.05% |
| Water | 26.00 | g | 98.60% |
| Potassium | 0.025 | g | 0.09% |
| sorbate | | | |
| Potassium | 0.005 | g | 0.02% |
| chloride | | | |
| Agar | 0.175 | g | 0.66% |
| Kappa | 0.100 | g | 0.38% |
| Carrageenan | | | |
| Sodium | 0.050 | g | 0.19% |
| alginate | | | |
| Citric acid | 0.0004 | g | 0.0015% |
| TOTAL | 26.3679 | g | 100.00% |

### Example 4 (Hydrochlorothiazide)

TABLE 8
Hydrochlorothiazide (12.5 mg/25 ml) - pH 7.5

| INGREDIENT | MASS | UNIT | % |
|---|---|---|---|
| Hydrochlorothiazide | 0.0125 | g | 0.05% |
| Water | 25.87 | g | 98.65% |
| Potassium sorbate | 0.025 | g | 0.10% |
| Potassium chloride | 0.005 | g | 0.02% |
| Magnesium chloride | 0.011 | g | 0.04% |
| Agar | 0.150 | g | 0.57% |
| Kappa Carrageenan | 0.100 | g | 0.38% |
| Sodium alginate | 0.050 | g | 0.19% |
| Citric acid | 0.0004 | g | 0.0015% |
| TOTAL | 26.2239 | g | 100.00% |

1. A drinkable gel formulation for a Biopharmaceutics Classification System (BCS) class II Active Pharmaceutical Ingredient (API), comprising:
i) a Biopharmaceutics Classification System (BCS) class II Active Pharmaceutical Ingredient (API), or a salt or solution thereof,
ii) a combination of gelling agents comprising:
a. a primary gelling agent which is agar,
b. a secondary gelling agent which is carrageenan, and
c. a tertiary gelling agent which is an alginate,
iii) at least one cation donator,
iv) a pH modifier,
v) a preservative, and
vi) water.

2. The drinkable gel formulation as claimed in claim 1, further comprising:
vii) a flavouring.

3. The drinkable gel formulation as claimed in claim 1, wherein
i) the API is ibuprofen, naproxen, diclofenac or hydrochlorothiazide, or a salt or solution thereof.

4. The drinkable gel formulation as claimed in claim 3, wherein
i) the ibuprofen, naproxen or diclofenac is a sodium salt or solution.

5. The drinkable gel formulation as claimed in claim 1, wherein
ii) a. the agar is agar-agar,
b. the carrageenan is kappa carrageenan, and
c. the alginate is sodium alginate.

6. The drinkable gel formulation as claimed in claim 1, wherein
iii) the at least one cation donator is one of a calcium, magnesium, or potassium salt.

7. The drinkable gel formulation as claimed in claim 6, wherein
iii) the at least one cation donator comprises magnesium and potassium chloride.

8. The drinkable gel formulation as claimed in claim 6, wherein
iii) the at least one cation donator consists of potassium chloride.

9. The drinkable gel formulation as claimed in claim 1, wherein
iv) the at least one pH modifier is citric acid.

10. The drinkable gel formulation as claimed in claim 1, wherein
v) the at least one preservative is potassium sorbate.

11. The drinkable gel formulation as claimed in claim 1, wherein
ii) the gelling agents are present in an amount percent by weight of:
a. agar-0.47%-0.95%
b. carrageenan-0.31%-0.47%, and
c. an alginate-0.16%-0.24%.

12. The drinkable gel formulation as claimed in claim 11, wherein
ii) the gelling agents are present in an amount percent by weight of:
a. agar-0.47%-0.71%
b. carrageenan-0.31%-0.47%, and
c. an alginate-0.16%-0.24%.

13. The drinkable gel formulation as claimed in claim 11, wherein
ii) the gelling agents are present in an amount percent by weight of:
a. agar-0.63%-0.95%
b. carrageenan-0.31%-0.47%, and
c. an alginate-0.16%-0.24%.

14. The drinkable gel formulation as claimed in claim 12, comprising excipients of Table 1 in the range as set out below:

| Excipient | Weight percent (Range) |
|---|---|
| Ibuprofen (sodium | 0.69-1.03% |
| salt) | |
| Water | ≥78.18% |
| Potassium sorbate | 0.078%-0.117% |
| Magnesium | 0.034%-0.052% |
| chloride | |
| Potassium chloride | 0.016%-0.024% |
| Agar | 0.47%-0.71% |
| Kappa | 0.31%-0.47%, |
| Carrageenan | |
| Sodium alginate | 0.16%-0.24%. |
| Citric acid | 0.066%-0.099% |

15. The drinkable gel formulation as claimed in claim 14, comprising by mean weight:

| INGREDIENT | MASS | UNIT |
|---|---|---|
| Ibuprofen | 0.200 | g |
| Water | 25 | g |
| Potassium | 0.025 | g |
| sorbate | | |
| Magnesium | 0.011 | g |
| chloride | | |
| Potassium | 0.005 | g |
| chloride | | |
| Agar | 0.150 | g |
| Kappa | 0.100 | g |
| Carrageenan | | |
| Sodium alginate | 0.050 | g |
| Citric acid | 0.021 | g |
| TOTAL | 25.562 | g |

or

| INGREDIENT | MASS | UNIT |
|---|---|---|
| Ibuprofen | 0.220 | g |
| Water | 25 | g |
| Potassium sorbate | 0.025 | g |
| Magnesium chloride | 0.011 | g |
| Potassium chloride | 0.005 | g |
| Agar | 0.150 | g |
| Kappa Carrageenan | 0.100 | g |
| Sodium alginate | 0.050 | g |
| Citric acid | 0.021 | g |
| TOTAL | 25.582 | g |

16. The drinkable gel formulation as claimed in claim 13, comprising excipients of Table 3 in the range as set out below:

| Excipient | Weight percent (Range) |
|---|---|
| Naproxen | 0.69%-1.03% |
| Water | ≥78.12% |
| Potassium sorbate | 0.08%-0.12% |
| Potassium | 0.16%-0.23% |
| chloride | |
| Agar | 0.63%-0.94% |
| Kappa | 0.31%-0.47% |
| Carrageenan | |
| Sodium alginate | 0.16%-0.23%. |
| Citric acid | 0.0009%-0.0014% |

17. The drinkable gel formulation as claimed in claim 16, comprising by mean weight the excipients of Table 4 as set out below:

| INGREDIENT | MASS | UNIT |
|---|---|---|
| Naproxen | 0.22 | g |
| Water | 25.00 | g |
| Potassium | 0.025 | g |
| sorbate | | |
| Potassium | 0.005 | g |
| chloride | | |
| Agar | 0.200 | g |
| Kappa | 0.100 | g |
| Carrageenan | | |
| Sodium alginate | 0.050 | g |
| Citric acid | 0.0003 | g |
| TOTAL | 25.60 | g |

18. The drinkable gel formulation as claimed in claim 11, comprising excipients of Table 5 in the range as set out below:

| Excipient | Weight percent (Range) |
|---|---|
| Diclofenac | 0.038%-0.057% |
| Water | >78.88% |
| Potassium sorbate | 0.076-0.114% |
| Potassium chloride | 0.015-0.023% |
| Agar | 0.53-0.79% |
| Kappa Carrageenan | 0.30-0.46% |
| Sodium alginate | 0.15-0.23% |
| Citric acid | 0.0012-0.0018% |

19. The drinkable gel formulation as claimed in claim 18, comprising by mean weight the excipients of Table 6 as set out below:

| INGREDIENT | MASS | UNIT |
|---|---|---|
| Diclofenac | 0.0125 | g |
| Water | 26.00 | g |
| Potassium | 0.025 | g |
| sorbate | | |
| Potassium | 0.005 | g |
| chloride | | |
| Agar | 0.175 | g |
| Kappa | 0.100 | g |
| Carrageenan | | |
| Sodium | 0.050 | g |
| alginate | | |
| Citric acid | 0.0004 | g |
| TOTAL | 26.3679 | g |

20. The drinkable gel formulation as claimed in claim 11, comprising excipients of Table 7 in the range as set out below:

| Excipient | Weight percent (Range) |
|---|---|
| Hydrochlorothiazide | 0.038-0.057% |
| Water | ≥78.9% |
| Potassium sorbate | 0.076-0.11% |
| Potassium chloride | 0.015-0.023% |
| Magnesium chloride | 0.034-0.050% |
| Agar | 0.46-0.69% |
| Kappa Carrageenan | 0.31-0.46% |
| Sodium alginate | 0.15-0.23% |
| Citric acid | 0.0012-0.0018% |

21. The drinkable gel formulation as claimed in claim 20, comprising by mean weight the excipients of Table 8 as set out below:

| INGREDIENT | MASS | UNIT |
|---|---|---|
| Hydrochlorothiazide | 0.0125 | g |
| Water | 25.87 | g |
| Potassium sorbate | 0.025 | g |
| Potassium chloride | 0.005 | g |
| Magnesium chloride | 0.011 | g |
| Agar | 0.150 | g |
| Kappa Carrageenan | 0.100 | g |
| Sodium alginate | 0.050 | g |
| Citric acid | 0.0004 | g |
| TOTAL | 26.2239 | g |
